SEQUENCE LISTING (16 sequences)

SEQ ID NO: 1 - DNA, 82 bp, Artificial Sequence
  Padlock Probe RPP 30; PO4 on 5′ end
  misc_feature 1: /note=“PO4 on the 5′ end”
ttgttgagtgttggcgtgtatgcagctcctcagtaatagtgtcttacatttagcatacatcgtcgcgtgcataaccaggcca

SEQ ID NO: 2 - DNA, 25 bp, Artificial Sequence
  Detection Oligonucleotide; Cy-3 on the 3′ end
  misc_feature 25: /note=“Cy3 on the 3′ end”
tcctcagtaatagtgtcttactttt

SEQ ID NO: 3 - DNA, 82 bp, Artificial Sequence
  PADLOCK PROBE GAPDH; PO4 on 5′ end
  misc_feature 1: /note=“PO4 on the 5′ end”
aagggtgcagctgaggtgtatgcagctcctcagtaatagtgtcttacatttagcatacatcgtcgcgagcgttttctcccta

SEQ ID NO: 4 - DNA, 82 bp, Artificial Sequence
  PADLOCK PROBE NRXN1 UNEDITED; PO4 on 5′ end
  misc_feature 1: /note=“PO4 on the 5′ end”
cggcggccgcctgcagtgtatgcagctcctcagtaatagtgtcttacgggccttattccggtgctatgctgattctgacgcg

SEQ ID NO: 5 - DNA, 82 bp, Artificial Sequence
  PADLOCK PROBE NRXN1 REFERENCE; PO4 on 5′ end
  misc_feature 1: /note=“PO4 on the 5′ end”
aataagggtcccgaggtgtatgcagctcctcagtaatagtgtcttacagagagtagtacttccgactacaccgtgacgaaga

SEQ ID NO: 6 - DNA, 25 bp, Artificial Sequence
  CAPTURE OLIGONUCLEOTIDE; biotin on 5′ end
  misc_feature 1: /note=“Biotin on the 5′ end”
tttttcctcagtaatagtgtcttac

SEQ ID NO: 7 - DNA, 20 bp, Artificial Sequence
  DETECTION OLIGONUCLEOTIDE CY3; Cy-3 on the 3′ end
  misc_feature 20: /note=“Cy3 on the 3′ end”
atttagcatacatcgtcgcg

SEQ ID NO: 8 - DNA, 25 bp, Artificial Sequence
  DETECTION OLIGONUCLEOTIDE FITC; FITC on the 3′ end
  misc_feature 25: /note=“FITC on the 3′ end”
gggccttattccggtgctatttttt

SEQ ID NO: 9 - DNA, 82 bp, Artificial Sequence
  PADLOCK PROBE ACTB REFERENCE; PO4 on 5′ end
  misc_feature 1: /note=“PO4 on the 5′ end”
ttgtagaaggtgtgggtgtatgcagctcctcagtaatagtgtcttacgggccttattccggtgctatgccacacgcagctca

SEQ ID NO: 10 - DNA, 82 bp, Artificial Sequence
  PADLOCK PROBE ACTB EDITED; PO4 on 5′ end
  misc_feature 1: /note=“PO4 on 5′ end”
ccttcaccgttccaggtgtatgcagctcctcagtaatagtgtcttacatttagcatacatcgtcgcgaaccgactgctgtca

SEQ ID NO: 11 - DNA, 82 bp, Artificial Sequence
  PADLOCK PROBE NRXN1 SNP; PO4 on 5′ end
  misc_feature 1: /note=“PO4 on 5′ end”
cggcggccgcctgcagtgtatgcagctcctcagtaatagtgtcttacgggccttattccggtgctatgctgattctgacgct

SEQ ID NO: 12 - DNA, 82 bp, Artificial Sequence
  PADLOCK PROBE ME28SA2486 REFERENCE; PO4 on 5′ end
  misc_feature 1: /note=“PO4 on 5′ end”
tcaacaccgctggcagtgtatgcagctcctcagtaatagtgtcttacgggccttattccggtgctatcggagacagagagct

SEQ ID NO: 13 - DNA, 82 bp, Artificial Sequence
  PADLOCK PROBE ACTB REFERENCE 2; PO4 on 5′ end
  misc_feature 1: /note=“PO4 on 5′ end”
ttgtagaaggtgtgggtgtatgcagctcctcagtaatagtgtcttacagagagtagtacttccgactgccacacgcagctca

SEQ ID NO: 14 - DNA, 82 bp, Artificial Sequence
  PADLOCK PROBE ACTB REFERENCE MUT; PO4 on 5′ end
  misc_feature 1: /note=“PO4 on 5′ end”
ttgtagaaggtgtgggtgtatgcagctcctcagtaatagtgtcttacatttagcatacatcgtcgcggccacacgcagctcg

SEQ ID NO: 15 - DNA, 20 bp, Artificial Sequence
  DETECTION OLIGONUCLEOTIDE ATTO647; ATTO647 on the 3′ end
  misc_feature 20: /note=“ATTO647 on the 3′ end”
agagagtagtacttccgact

SEQ ID NO: 16 - DNA, 20 bp, Artificial Sequence
  EXTERNAL PRIMER; bases in positions 19 and 20 each have a phosphorotiaoate modification
  misc_feature 19..20: /note=“bases in positions 19 and 20 each have a phosphorotiaoate modification”
tactgaggagctgcatacac

(19) United States
(12) Patent Application Publication
(10) Pub. No.: US 2024/0158842 A1
(43) Pub. Date: May 16, 2024

(54) METHODS AND USES FOR DETERMINING THE EFFICIENCY OF GENETIC-EDITING PROCEDURES

(71) Applicant: COUNTAGEN AB, Solna (SE)

(72) Inventors: Felix NEUMANN, Stockholm (SE); Leiore Ajuria ASTOBIZA, Stockholm (SE); Joost BERGMAN, Stockholm (SE)

(21) Appl. No.: 18/282,017

(22) PCT Filed: Mar. 15, 2022
(86) PCT No.: PCT/EP2022/056740

§ 371 Date: Sep. 14, 2023

(30) Foreign Application Priority Data
Mar. 15, 2021 (GB) 2103543.1

(51) Int. Cl. C12Q 1/6851; C12Q 1/6858; C12Q 1/6876

(52) U.S. Cl. CPC C12Q 1/6851; C12Q 1/6858; C12Q 1/6876

(57) ABSTRACT

The invention relates to methods and uses for determining the efficiency of a genetic editing procedure comprising the steps of: (i) providing a sample from a genetic-editing procedure, the sample comprising one or more correctly-edited polynucleotide sequence and/or one or more unedited polynucleotide sequence; (ii) performing Rolling Circle Amplification, to generate RCA-Products from the one or more polynucleotide sequences in the sample; and (iii) determining the efficiency of the genetic-editing procedure based on the presence of the RCA-Products generated in step (ii).

The present invention relates to methods and uses for determining the efficiency of genetic-editing procedure and/or identifying the products of genetic-editing procedures.

The possibility to perform targeted genetic modifications with high efficiency is a key step in drug discovery, personalized medicine, and agriculture productivity.

Genetic-editing has created many breakthroughs as it allows precise genome engineering using special nucleases. Numerous genetic-editing tools have been developed, including zinc finger nucleases (ZFNs), transcription activator-like effector nucleases (TALENs), and the now-popular CRISPR/Cas9-based systems (CRISPR, clustered regularly interspaced short palindromic repeats; Cas9, CRISPR-associated protein 9). Many of those tools and systems are now widely used and are technically simple to perform.

However, despite their practical simplicity, the efficiency of such systems can be variable, and result in a low proportion of correctly-edited genetic products. For example, the efficiency of CRISPR/Cas9 systems is dependent on the design of the single guide RNA (“sgRNA”) that is used. To ensure maximum editing efficiency using CRISPR-based systems, it is therefore necessary to carefully screen and optimize the sgRNA that is used.

A critical step in the development of engineering genomes is to prescreen efficiency and specificity of the used system. Accurate quantification and identification of targeted genetic-editing events is therefore critical for the development, characterization and wider application of genetic-editing techniques.

A range of approaches for detecting and quantifying the results of genetic-editing procedures have been developed.

Current strategies to screen and validate editing efficiency are based on Polymerase Chain Reaction (“PCR”), a multi-step method which requires specialist laboratory equipment to perform. PCR is used in Sanger and Next-Generation Sequencing and, whilst those techniques do provide accuracy with a high resolution, they require further complex and expensive sequencing equipment. Digital droplet PCR (“ddPCR”) provides high accuracy but its implementation is hindered by the cost of instrumentation and the tedious optimisation steps required to achieve optimal results before it can be used. The PCR-based “T7 Endonuclease I” or “Surveyor” screening approach is technically simpler than sequencing and ddPCR but suffers from low accuracy. These methods are described in more detail below.

The most accurate but also most time consuming (often taking days) and most expensive methods are sequencing-based; these mainly include Sanger sequencing and NGS. Sanger sequencing requires the input of a single clone, which increases the amount of work required for this type of method. New in silico methods like ‘tracking of indels by decomposition’ (TIDE) allow the analysis of mixed cell populations (Brinkman, E. K.; Chen, T.; Amendola, M.; Van Steensel, B. Easy Quantitative Assessment of Genome Editing by Sequence Trace Decomposition. Nucleic Acids Res. 2014, 42 (22), e168).

Sanger sequencing in conjunction with TIDE requires high quality DNA and reads, as otherwise errors occur easily. NGS can perform highly multiplexed deep sequencing to identify rare mutations in cell populations which go introduced by gene editing (Schmitt, M. W.; Kennedy, S. R.; Salk, J. J.; Fox, E. J.; Hiatt, J. B.; Loeb, L. A. Detection of Ultra-Rare Mutations by next-Generation Sequencing. Proc. Natl. Acad. Sci. U.S.A 2012, 109 (36), 14508-14513). Nevertheless, its main drawbacks are the error rate, cost per run making it uneconomic as a prescreening tool for gene editing experiments.

The ddPCR method allows more-precise measures of the efficiency of the gene editing event, and makes use of fluorescent probes for quantification (see, for example, Miyaoka, Y.; Mayerl, S. J.; Chan, A. H.; Conklin, B. R. Detection and Quantification of HDR and NHEJ Induced by Genome Editing at Endogenous Gene Loci Using Droplet Digital PCR. In Methods in Molecular Biology; Humana Press Inc., 2018; Vol. 1768, pp 349-362). The drawback of ddPCR is the need for specialized instruments to perform the digitizing of target DNA into droplets, to run the droplet PCR reaction and finally to read out the result in a droplet reader.

Denaturation-based methods are considered as the most low-tech solution as they do not require any specialized instrumentation from the perspective of a standard molecular biology laboratory. However, the information content provided by these methods is very limited as they are solely based on differences of the edited to unedited genome (Germini, D.; Tsfasman, T.; Zakharova, V. V.; Sjakste, N.; Lipinski, M.; Vassetzky, Y. A Comparison of Techniques to Evaluate the Effectiveness of Genome Editing. Trends in Biotechnology. Elsevier Ltd Feb. 1, 2018, pp 147-159). These differences are most often measured by band intensity after a gel electrophoresis, such as in the “T7 endonuclease I” or “Surveyor” assay (Vouillot, L.; Thélie, A.; Pollet, N. Comparison of T7E1 and Surveyor Mismatch Cleavage Assays to Detect Mutations Triggered by Engineered Nucleases. G3 Genes, Genomes, Genet. 2015, 5 (3), 407-415.). Therefore, those methods can only estimate the editing activity without giving information on the gene editing efficiency.

FIG. 1 depicts the differences in workflow between those methods. A more detailed comparison of different available methods can be found in (Germini, D.; Tsfasman, T.; Zakharova, V. V.; Sjakste, N.; Lipinski, M.; Vassetzky, Y. A Comparison of Techniques to Evaluate the Effectiveness of Genome Editing. Trends in Biotechnology. Elsevier Ltd Feb. 1, 2018, pp 147-159).

Against this background, the present inventors have developed an improved approach for identifying the products of genetic-editing procedures and determining the efficiency of such procedures. The approach is also useful in determining the specificity of the gene-editing procedures.

Unlike the prior art techniques, the inventors' approach does not rely on PCR-based detection, and it therefore avoids the multiple steps and expensive and specialist laboratory equipment that are required to perform PCR and subsequent steps, such as reading droplets in ddPCR or sending PCR amplicons for sequencing to external services. As explained below and in the accompanying examples, despite its simplicity, the inventors' technique can rapidly and accurately detect the products of genetic-editing procedures and determine the efficiency of such procedures.

In a first aspect, the invention provides a method for determining the efficiency of a genetic-editing procedure, the method comprising the steps of:
(i) providing a sample from a genetic-editing procedure, the sample comprising one or more correctly-edited polynucleotide sequence and/or one or more unedited polynucleotide sequence;
(ii) performing Rolling Circle Amplification, to generate RCA-Products from the one or more polynucleotide sequences in the sample; and
(iii) determining the efficiency of the genetic-editing procedure based on the presence of the RCA-Products generated in step (ii).

Thus, unlike the prior art based approaches, the inventors' method uses Rolling Circle Amplification (“RCA”) as a tool to analyse the polynucleotide sequences that are produced by the genetic-editing procedure. Specifically, following the genetic-editing procedure, RCA is used to selectively generate RCA-Products (also referred to herein as “RCA products”, “RCP” or “RCPs”) from the correctly-edited polynucleotide sequences and from the unedited polynucleotide sequences that have been produced. After RCA the RCA-products are present in a liquid sample, which can undergo further processing steps to analyse the RCA-products, either as a liquid sample or as a dry solid sample.

RCA is a well-known single molecule amplification method that allows for digital quantification without compartmentalization. After labeling RCA products with molecules of defined optical properties such as fluorophores, said amplified molecules can be detected as single dots that can be quantified individually. Circular oligonucleotide templates to perform RCA can be designed and produced by a number of highly target-specific means, and these targets can be virtually any nucleotide sequence. By tailoring the assay to detect edited and unedited variants from genomic material that has been subjected to a gene editing technique (for example, CRISPR/Cas9) it is possible to estimate its efficiency in a simple, yet precise manner.

RCA uses highly processive polymerases on a circular DNA target to generate a long ssDNA (i.e. single-stranded DNA) concatemer in hundreds of nanometers- to micrometer-range (Banér, J.; Nilsson, M.; Mendel-Hartvig, M.; Landegren, U. Signal Amplification of Padlock Probes by Rolling Circle Replication. Nucleic Acids Res. 1998, 26 (22), 5073-5078). RCA is often combined with “padlock probes” (PLPs), sequence specific oligonucleotides binding in a circular manner to the target strand which can then be covalently linked by a ligation step. A PLP-based RCA assay offers extreme stringency with single base precision (Nilsson, M.; Malmgren, H.; Samiotaki, M.; Kwiatkowski, M.; Chowdhary, B. P.; Landegren, U. Padlock Probes: Circularizing Oligonucleotides for Localized DNA Detection. Science. 1994, 265 (5181), 2085-2088). Similar to PLPs, “selector” probes can be combined with RCA, where the target is circularized prior to RCA (Johansson, H.; Isaksson, M.; Sörqvist, E. F.; Roos, F.; Stenberg, J.; Sjöblom, T.; Botling, J.; Micke, P.; Edlund, K.; Fredriksson, S.; Kultima, H. G.; Ericsson, O.; Nilsson, M. Targeted Resequencing of Candidate Genes Using Selector Probes. Nucleic Acids Res. 2011, 39 (2), e8).

Yet another probe mechanism uses molecular inversion probes which have been demonstrated in highly multiplexed assays for single nucleotide polymorphism detection as well as for genotyping (Antson, D. O.; Isaksson, A.; Landegren, U.; Nilsson, M. PCR-Generated Padlock Probes Detect Single Nucleotide Variation in Genomic DNA. Nucleic Acids Res. 2000, 28 (12), e58; and Krishnakumar, S.; Zheng, J.; Wilhelmy, J.; Faham, M.; Mindrinos, M.; Davis, R. A Comprehensive Assay for Targeted Multiplex Amplification of Human DNA Sequences. Proc. Natl. Acad. Sci. U.S.A 2008, 105 (27), 9296-9301). Most commonly used ligases for sealing the nick of aforementioned probes are T4 DNA and Tth ligase for ligation on DNA, and T4 RNA and SplintR ligases for ligation on RNA.

For amplification, phi29 DNA polymerase is most often as it has an extremely high processivity as well as a 3′ to 5′ proofreading exonuclease activity. RCA has been applied to cancer profiling (Huang, R.; He, L.; Li, S.; Liu, H.; Jin, L.; Chen, Z.; Zhao, Y.; Li, Z.; Deng, Y.; He, N. A Simple Fluorescence Aptasensor for Gastric Cancer Exosome Detection Based on Branched Rolling Circle Amplification. Nanoscale 2020, 12 (4), 2445-2451), and the detection of pathogens (Neumann, F.; Hernández-Neuta, I.; Grabbe, M.; Madaboosi, N.; Albert, J.; Nilsson, M. Padlock Probe Assay for Detection and Subtyping of Seasonal Influenza. Clin. Chem. 2018, clinchem. 2018.292979) for the potential use in clinical diagnostics.

However, to date, RCA/probe-based assays have not been used in the for the screening of genetic editing. The present inventors' approach is the first to do so, and results in a high specificity and a simple approach for doing so.

The presentation of the currently available methods clearly highlights the need for new methods that provide the ease and requirements of a denaturation-based method while offering the same or more information content as ddPCR in a ready-to-use fashion.

That is provided by the present invention, which makes use of RCA to allow for the precise determination of gene editing efficiency. The present invention cuts down time and costs spend for gene editing analysis from days to just a few hours. Furthermore, the generated RCPs can be used in enrichment methods as well as be used in sequencing applications if required. Benefits of the RCA-based approach of the invention include that no digitizing is required as one DNA target corresponds to one RCP; it is highly specific due to the use of probes that encompass two binding arms; it enables multiplexed reactions without the need for switching fluorescent probes as probe backbones can remain constant while the arms are target specific; and, it does not require any specialized instrumentation unlike ddPCR and sequencing based methods as RCA can be performed with standard equipment and be readily readout under a fluorescence microscope.

Despite being well known, RCA has never been used in the context of the method of the invention. Instead, as is evident from the discussion above, those in the field were focused on developing ever-more complex ways of detecting gene-editing events, such as ddPCR and DNA-sequencing based approaches.

The inventors' approach is therefore conceptually different to the direction of developments in the field is developing and does not follow logically from the art. The invention allows for a better way to determine the efficiency and overall products of the genetic-editing procedure and, as shown in the examples and herein, provides a number of surprising advantages when compared to current methods, including:
Simplicity of use;
Low cost;
Shorter turn-around time;
Higher sensitivity;
Higher specificity; and
The possibility of using standard laboratory equipment rather than specialized technologies.

This improved method of the invention is, therefore, an important development in the field.

Step (i) of the method of the invention involves providing a sample from a genetic-editing procedure. By “genetic-editing procedure” we include any such procedure in which a target polynucleotide sequence is “edited” by addition and/or deletion and/or mutation of one or more nucleotide base in that molecule. The term “genetic-editing” also encompasses “genome-editing” and “gene-editing”. Several genome editing tools have been developed, including ZFNs, TALENs, and the now popular CRISPR/Cas9 system, and each of these editing tools are included under what is referred to herein as “genetic-editing procedure”.

Genetic-editing procedures allow for genetic material in a polynucleotide sequence to be added, removed, or altered at particular locations. Thus, from “a sample from a genetic-editing procedure” we include a sample containing the resulting product of the genome editing procedure. As will be appreciated, such a sample may contain one or more correctly-edited polynucleotide sequence and/or one or more unedited polynucleotide sequence. Furthermore, the sample may also contain one or more incorrectly edited polynucleotide sequence.

For the avoidance of doubt, it is contemplated within the scope of the invention that the method may be used to determine whether the sample contains polynucleotide sequences that have been 100% correctly edited, or polynucleotide sequences that have been 100% non-edited (i.e. 0% non-edited), or polynucleotide sequences that have been 100% incorrectly edited, or any combination thereof.

By “polynucleotide sequence” we include any biopolymer composed of nucleotide monomers in a chain, for example DNA and/or cDNA and/or RNA. Each polynucleotide sequence may be present in the sample in an amount of from about from about 0.1 ng to about 5000 ng, such as from about 0.1 ng to about 2000 ng, for example from about 1 ng to about 1000 ng. Put another way, in step (ii) each polynucleotide sequence may be present in an amount of from about 0.01 ng/μL to 500 ng/μL, such as from about 0.05 ng/μL to about 250 ng/μL.

Step (ii) of the method of the invention requires performing Rolling Circle Amplification, to generate RCA-Products from the one or more polynucleotide sequences in the sample.

In an embodiment the method comprises the step of preparing the sample prior to RCA, wherein the preparation steps include DNA extraction from the sample, denaturation of the DNA, followed by probe hydbridisation and ligation.

In relation to step (iii), it will be appreciated that any single molecule detection method can be used. Such single molecule detection methods may include fluorescence microscopy, such as epifluorescence microscopy.

Preferably in the method of the invention, the RCA-Products are labelled with a detectable moiety.

In an embodiment the detectable moiety can be selected from the list consisting of fluorophores, chromophores and nanoparticles, preferably fluorophores.

In an embodiment the detectable moiety can is a spectrally separated fluorophore selected from the list consisting of Cyanine 3, Cyanine 5, Alexa Fluor family dyes (such as 488 and 750), fluorescein (FITC), Atto family dyes (such as ATTO 550 and ATTO 488), quantum dots, and synthetic fluorophores.

In a preferred embodiment, different detectable moieties are used to label the RCA-Products generated from the correctly-edited polynucleotide sequence and the RCA-Products generated from the unedited polynucleotide sequence. In that embodiment of the invention, the RCA-Products are therefore differentially-labelled, which allows RCA-Products from the correctly-edited polynucleotide sequence and/or the unedited polynucleotide sequence to be distinguished from one another.

Furthermore, an additional detectable moiety may be used to label incorrectly edited polynucleotide sequences in the sample, wherein this datable moiety is different to the moieties used to label the correctly-edited and unedited polynucleotide sequences.

Preferably in the method of the invention, step (iii) comprises quantifying the RCA-Products generated in step (ii), and determining the amounts of the correctly-edited polynucleotide sequence and/or unedited polynucleotide sequence in the sample. Correctly-edited and unedited polynucleotide sequences may be quantified relative to one another and/or may be quantified relative to the total number of polynucleotide sequences in the sample by also quantifying the total number of incorrectly-edited polynucleotide sequences.

In this embodiment where the method of the invention enables the accurate quantification of targeted gene editing efficiency and identification of the same by using RCA, the method may also use gene editing site specific circularized oligonucleotides sequences.

In an embodiment the RCA-products may be differentially labeled by hybridizing labeled oligonucleotides complementary to the control, unedited gene sequence and edited sequence and the level of hybridisation allows for the sequences to be quantified and this in turn allows for the ratio of editing efficiency to be determined.

Oligonucleotides can be labeled by various reporter molecules including but not limited to fluorophores, chemiluminescent labels, colorimetric labels, phosphorescent labels and particles, such as quantum dots, gold particles, or silver particles.

In an embodiment, individual circularizable probes or circularized gene targets are designed or selected to comprise edited and unedited gene sites. Said probes or targets can be ligated into circles that are substrates for an RCA reaction. Resulting amplicons from individual reacted probes can be differentially labeled and quantified in a digital manner. By measuring the ratio of amplicons coming from a control/reference, edited and unedited target site probes, it is possible to calculate the efficiency of the gene editing technique as well as identify the edit itself with high precision.

By “quantifying the RCA-Products” we include quantifying in a digital manner.

In a particular embodiment, resulting RCA-products can be detected and quantified by single molecule detection schemes that allow for differential detection, including but not limited to, amplified single-molecule detection, by immobilisation on a solid support such as a microarray, or enrichment on a filter membrane.

Quantification is achieved by imaging immobilized RCPs with a fluorescence microscope. Resulting images are processed with an image processing software that performs top-hat filtering, spot registration, spot size filtering and spot quantification algorithms. Where, each spot corresponds to a resulting RCP from a target molecule. This counting of individual spots or positive entities is what we called digital quantification.

In an embodiment the number of RCPs in the sample is greater than 1 copy, such as greater than 10, 100, 200, 400 or 500 copies.

In a preferred embodiment, the method further comprises the step of quantifying the total number of polynucleotide sequences in the sample.

By “total number of polynucleotide sequences in the sample” we include the combined total number of correctly-edited polynucleotide sequences, unedited polynucleotide sequences and incorrectly-edited sequences in the sample.

In an embodiment, the total number of correctly-edited polynucleotide sequences may be quantified followed by quantifying the total number of unedited polynucleotide sequences, quantifying the total number of correctly-edited polynucleotide sequences and quantifying the total number of incorrectly-edited polynucleotide sequences, and totaling the amounts. The quantification of each sequence type may be done in any order. Alternatively, the total number of correctly-edited polynucleotide sequences, unedited polynucleotide sequences and incorrectly-edited polynucleotide sequences can be quantified and totaled simultaneously.

Preferably, step (ii) of the method comprises generating circular single-stranded polynucleotide substrates from the polynucleotide sequences in the sample, and wherein the circular single-stranded polynucleotide substrates are specific for the correctly-edited polynucleotide sequence, and for the unedited polynucleotide sequence.

By “circular single-stranded polynucleotide substrates” we include the substrates produced using the probes described herein followed by probe hybridization and ligation.

More preferably, the circular single-stranded polynucleotide substrates are generated using a first oligonucleotide probe which specifically targets the correctly-edited polynucleotide sequence, and a second oligonucleotide probe which specifically targets the unedited polynucleotide sequence. This is generally achieved by using probes that target the edited and non-edited sites that become circular only once they find their respective target.

It is preferred that the first and/or second oligonucleotide probes are selected from the group comprising: a padlock probe; a molecular inversion probe; a gap-fill probe; a split-like probe; a Lotus probe; a trilock probe or a combination thereof.

In an embodiment the first and/or second oligonucleotide probes are a combination of padlock probes which are advantageous for non-edited detection and oligonucleotide gap fill probes which are advantageous for edited detection.

As used herein the term “padlock probe” refers to single stranded DNA molecules with two segments complementary to the target connected by a linker polynucleotide sequence. When the complementary segments hybridise to the DNA target, the padlock probes become circularized.

In an embodiment, circular oligonucleotides are generated by the specific hybridization and ligation of padlock probes on their respective targets. Wherein, said probes are designed to contain differential sequences that allow to differentiate between the control, edited and the unedited gene sequences. After subsequent RCA reaction, multiple complementary copies of the original probe are generated. The resulting single molecule amplicons are differentially labeled by hybridizing labeled oligonucleotide tags corresponding to those originally encoded within each probe, which generates populations of differentially labeled amplicons coming from the control, edited and unedited gene sequences site, respectively.

By the term “molecular inversion probe” we refer to padlock probes that have been modified so that when the probe is hybridized to the polynucleotide fragment, a circular structure is formed with the intended target captured in a gap between the complementary segments.

As used herein, the term “gap-fill probe” relates to probes which have probe arms that are designed to flank the gene editing site after hybridization, thus leaving a gap on the editing site.

In an embodiment, circular substrates for RCA are generated by the specific hybridization and ligation of oligonucleotide gap-fill probes on their respective targets. The gap on the editing site is filled by hybridization and ligation of oligonucleotides complementary to the sequence contained within the gap thus forming circular oligonucleotide molecules. After subsequent RCA reaction, multiple complementary copies of the filled probe are generated. The resulting single molecule amplicons are differentially labeled by hybridizing labeled oligonucleotides complementary to those that originally fill the gap and to the joint arms of the Gap-fill probe, where gap filled labeled events will correspond to the unedited gene sequence and non-filled events will correspond to edited sequence.

By the term “split-like probe” we refer to probes that consists of at least two separate probes that have been designed in a way to be partially complementary to the polynucleotide target and to a connector sequence. Upon hybridization to the polynucleotide fragment, the probes come into close proximity and their backbone gets linked by the connector sequence. The nick in the backbone can be closed via a ligase while the nick or gap in the polynucleotide target site can be either closed by a ligase alone or in conjunction with a polymerase, respectively.

By the term “Lotus probe” we refer to probes that are already partially circular but require the specific hybridization of a polynucleotide target sequence to form a functional circular oligonucleotide molecule. The hybridized target fragment can either create two nick sites or a gap that can be subsequently closed by a ligase or in the case of the gap in conjunction with a polymerase. These probes are dumbbell shaped and consist either of a single probe or multiple separate probes.

By the term “Trilock probe” we refer to probes that consist of four separate probes a common backbone probe, two probes partially complementary to the polynucleotide target and a “trilock linker” which brings them all together by being partially complementary to the other probes. The two probe arms complementary to the polynucleotide target can either be perfectly matching to create a nick site that can be sealed with a ligase, or create a gap that is filled by a polymerase before being sealed by a ligase.

By the term “sequencing probe” we refer to probes that consist barcode(s) in the backbone to enable sequencing of the evolving RCPs by being compatible with NGS chemistry.

In an embodiment, the first and second oligonucleotide probes circularise on recognition of their specific target polynucleotide sequence.

Circularisation occurs when the ends of the polynucleotide sequence (these can be a probe or a target) hybridize next to each other forming the circle. Then a DNA ligase joins the ends closing completely the circular molecule. For gap fill probes (aka molecular inversion probes) circularization happens after a polymerase fills the gap in between the ends of the probe and then a ligase joins the ends.

In a preferred embodiment, circularisation of the first and/or second oligonucleotide probes is mediated and/or improved by one or more Joining probe; optionally wherein the one or more Joining probe is a Selector probe.

Circular substrates may be generated by circularizing the gene editing site region, wherein polynucleotide sequences from the genetic-editing procedure are fragmented by, for instance, restriction endonucleases and are then subsequently denatured. A so-called selector probe, complementary to the joint ends of the fragmented region, may be used to circularize the gene-edited region. After exonucleolytic digestion of any non-circular targets, RCA is performed thus generating multiple copies of the gene-edited polynucleotide sequence.

By “joining probe” we include probes that hybridize next to each other in a tail to end fashion and after ligation form a single DNA sequence strand.

In a preferred embodiment, the circular single-stranded polynucleotide substrates are formed by ligation of the first oligonucleotide probe and its specific target polynucleotide sequence, and by ligation of the second oligonucleotide probe and its specific target polynucleotide sequence; optionally wherein ligation is performed by a ligase with specific intramolecular ligation activity.

In a preferred embodiment, Rolling Circle Amplification of the circular single-stranded polynucleotide substrates is initiated by the target sequence or by an amplification primer that is complementary to the circular singe-stranded polynucleotide substrate.

In an embodiment, the polymerase that performs RCA requires a 3′OH end to initiate RCA. This 3′ end base may be complementary to the circle so it uses the circle as a template. When detecting sequences in genomic DNA, this 3′ end is not complementary to the circle, there is rather a long stretch of single stranded DNA attached to the circle. This results in two ways of starting the RCA.

When RCA is initiated by the target: The polymerase itself digests the long DNA stretch until it reaches the circle, once it reaches it finds the 3′end complementary to the circle and then it can initiate RCA. When RCA is initiated by a primer: A primer is hybridized to the circle and this hybridization produces this 3′ end and then the RCA can start.

By “amplification primer” we include a polynucleotide sequence that is fully complementary to the circular template.

In an embodiment the primer can be modified with a moiety to protect it from exonuclease activity. Such moieties can be selected from the group consisting of ortho methyl RNA bases and alpha-thiol phosphate linkages.

Preferably, the detectable moiety is selected from the group comprising: a fluorophore; a chromophore; or a combination thereof.

As outlined above, after the RCA step the RCA-products are present in a liquid sample. In a particularly preferred embodiment of the invention, step (iii) comprises immobilizing the RCA-Products on a surface.

For example, the RCA-products may be immobilized on the surface by electrostatic interactions, covalent interactions, or steric interactions with said surface.

Alternatively, the RCA-products be attached to magnetic beads to provide bead-bound RCA-products, and to immobilize the RCA-products on a surface a magnetic source may be provided so as to attract the bead-bound RCA-products on said surface.

The magnetic beads may have an average size of from about 10 nm to about 5 μm, for example from about 10 nm to about 2 μm, such as about 500 nm to about 2 μm. In this regard, the magnetic beads may have an average diameter from about 10 nm to about 5 μm, for example from about 10 nm to about 2 μm, such as about 500 nm to about 2 μm or about 50 nm to about 200 nm.

In another embodiment the surface is a glass surface or a porous membrane.

In an embodiment the porous membrane is a filter membrane, for example a porous hydrophilic membrane.

In an embodiment the RCA-Products are immobilized by filtering the liquid sample through the porous membrane. The liquid sample may be drawn through the porous membrane by gravity filtration, by applying a vacuum pump, or by capillary forces.

In an embodiment, the liquid sample is drawing through the porous membrane by capillary forces by applying the liquid sample to one side of the porous membrane and applying an absorption layer to the other side of the porous membrane to put the absorption layer and porous membrane into liquid connection and such the liquid from the liquid sample.

For the avoidance of doubt, the porous membrane is permeable for the liquid in the liquid sample, but substantially impermeable to the RCA-products.

By the term “capillary force(s)” as used herein, this refers to the sucking or wicking of liquid through the porous membrane so as to immobilize the RCA-products on the surface of the membrane.

In an embodiment the area of the porous membrane corresponds to a single field of view of an optical sensing device.

In an embodiment the porous membrane has a thickness of from about 0.01 μm to about 100 μm, such as from about 0.05 μm to 0.5 μm, for example from about 0.07 μm to about 0.2 μm, or wherein the filter membrane has a thickness of about 0.1 μm.

In another embodiment, the porous membrane has a surface area of from about 2 to about 20 mm², such as from about 5 to about 15 mm², for example from about 5 to about 10 mm².

In a further embodiment, the porous membrane is substantially circular in shape, such as circular in shape, wherein the porous membrane has a diameter in the range of from about 0.1 to about 10 mm, such as from about 0.5 mm to about 10 mm, for example from about 1 mm to about 5 mm, or from about 1 mm to about 3 mm, or wherein the filter membrane is circular having a diameter of about 2 mm.

In an embodiment, when the RCA-products are immobilized on a glass surface, the glass surface is modified to interact with an RCA-Product.

In an embodiment the glass surface may be modified with positively charged homopolymers, for example poly-L-Lysine, poly-D-lysine or aminosilane.

RCA-products may also contain affinity molecules such as biotin by introducing biotin-modified dNTPs into the RCA reaction mix. Biotinylated RCA-products can be immobilized on a glass surface modified with streptavidin.

In another embodiment, the RCA-products in the liquid sample are flown through a microfluidic channel so as to concentrate the sample into a single view of a microscope. The liquid flow may be achieved through use of a pump system or the flow may be achieved passively. This concentrating step may be referred to as microfluidic enrichment.

The enrichment/immobilization of the RCA-products allows for lower amounts/concentrations of polynucleotides to be used compared to currently known procedures. Particular amounts/concentrations of polynucleotides that can be used are outlined above.

In a preferred embodiment of the invention, step (iii) comprises detecting the RCA-Products by microscopy; optionally wherein microscopy is selected from the group comprising: bright-field microscopy; fluorescence microscopy; or a combination thereof.

In a preferred embodiment of the invention, step (iii) comprises detecting the RCA-Products by DNA sequencing.

As used herein, the term “DNA sequencing” refers to sequencing techniques such as sequencing by ligation, sequencing by synthesis or sequencing by hybridization as well as Sanger or next-generation sequencing. Such methods are well known to those skilled in the art of molecular biology.

Preferably, the efficiency of the genetic-editing procedure is determined based on the relative amounts of the correctly-edited polynucleotide sequence and the unedited polynucleotide sequence in the sample.

For example, the number of correctly edited polynucleotide sequences can be determined relative to a control with the remainder relating to unedited sequences. Should the total amount of combined edited and non-edited sequences not equal 100%, this difference can be attributed to incorrect or non-expected edits.

In an embodiment the efficiency of the gene-editing procedure may be determined by adding a detectable moiety that targets the same polynucleotide that is being edited, but in a different position to that which is being edited.

This allows for accurate quantification of efficiency of the method as depending on the method a gene may be edited, but not by the right sequence. Therefore, the detectable moiety theoretically binds to all polynucleotide sequences in the sample as it binds to a different position on the polynucleotide to that which is being edited, thus allowing for the total number of sequences to be determined. The moieties targeting the edited and non-edited sequence will provide quantification of the total number of correctly-edited and non-edited sequences, whilst the difference between the total number of polynucleotide sequences and the combination of correctly-edited and non-edited sequences will allow quantification of the incorrectly edited sequences.

The inclusion of this control probe allows for very accurate analysis of efficiency without relying on other more expensive and complicated methods, such as ddPCR and qPCR.

In a further aspect, the invention provides a method for identifying the product of a genetic-editing procedure, the method comprising the steps of:
(i) providing a sample from a genetic-editing procedure, the sample comprising one or more correctly-edited polynucleotide sequence and/or one or more unedited polynucleotide sequence;
(ii) performing Rolling Circle Amplification, to generate RCA-Products from the one or more polynucleotide sequences in the sample; and
(iii) determining the products of the genetic-editing procedure based on the presence of the RCA-Products generated in step (ii).

In a further aspect, the invention provides the use of Rolling Circle Amplification for identifying the product of a gene-editing procedure and/or for determining the efficiency of a genetic-editing procedure.

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

### BRIEF DESCRIPTION OF THE DRAWINGS

FIG. 1. Workflow comparison of the different methods available for prescreening gene editing efficiency and specificity.

FIG. 2. A schematic of the method to quantify editing efficiency and identify the edit by quantifying rolling circle amplicons.

FIG. 3. Schematics of the design and principle of generating circular templates for RCA using padlock probes targeting gene editing sites.

FIG. 4. Schematics of the design and principle of generating circular templates for RCA using Gap-fill probes targeting gene editing sites.

FIG. 5. Schematics of the design and principle of generating circular templates for RCA using selector probes targeting gene editing fragment sequences.

FIG. 6. Schematics of the design and principle of generating circular templates for RCA using splint probes, padlock probes, invader probes or splint probes for single nucleotide polymorphism sites.

FIG. 7. Schematics of the design and principle of generating circular templates for RCA using Lotus or Lotus-like probes targeting gene editing fragment sequences.

FIG. 8. Schematics of the design and principle of generating circular templates for RCA using triple split probes targeting gene editing fragment sequences.

FIG. 9. Schematics of the method to quantify editing efficiency and identify the edit by next generation sequencing of rolling circle amplification amplicons.

FIG. 10. RCPs generated in the presence and absence of a gene of interest. Only in the presence of a gene of interest RCPs are generated and can be seen as bright fluorescent entities under a fluorescence microscope. Scale bar=10 μm.

FIG. 11. Microfluidic enrichment of RCPs. Only in the sample with the gene of interest present can RCPs be observed under a fluorescence microscope. White arrows point to individual RCPs. Scale bar=10 μm.

FIG. 12. A: Exemplary images of regions of interest showing the multiplexed quantification of three genes. The composite images show all three colors, while the second-row shows the decomposed images for each channel and arrows indicating exemplary locations of RCPs. B: Graph illustrating the concept of digital quantifying 3 different genes with the same gene copy number using the described method. The average of two individual measurements is shown and the corresponding standard deviation.

FIG. 13. A: Graph illustrating the quantification of RCPs from samples with varying human DNA input and constant mouse DNA as control. B: Graph illustrating the data from A as a percentage of expected/theoretical versus the observed RCP ratio. A pure human DNA input was taken as the reference (100% theoretical and 100% observed) and the varying input related to that RCP count. The average of two individual measurements is shown and the corresponding standard deviation. C: Exemplary images of regions of interest showing the decreasing RCP quantity for decreasing human DNA input. The scale bar corresponds to 20 μm.

FIG. 14. A: Graph illustrating the specificity of the method to distinguish between single nucleotides using padlock probes and RCA. The average of two individual measurements is shown and the corresponding standard deviation. B: Exemplary regions of interest showing the presence of RCPs from a reference gene in both cases (Perfect Match and 1 nucleotide Mismatch) and the absence of RCPs in the case of a 1 nucleotide mismatch. The scale bar corresponds to 20 μm.

FIG. 15. Graph illustrating the capability of the method to quantify polynucleotide sequences from different organisms, as well as showing the padlock probe set up, where a reference shows the same level of events as the unedited sequence (compare Reference and Unedited bars). In case of an editing event, the ratio between reference to unedited or control to unedited allows to draw conclusions on the editing level. The average of two individual measurements is shown.

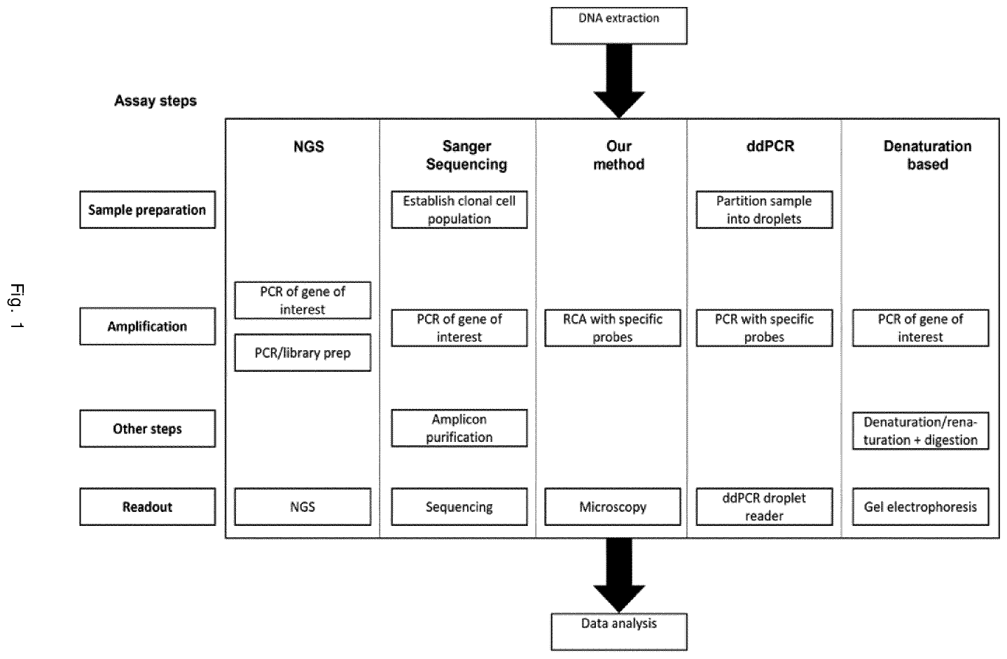

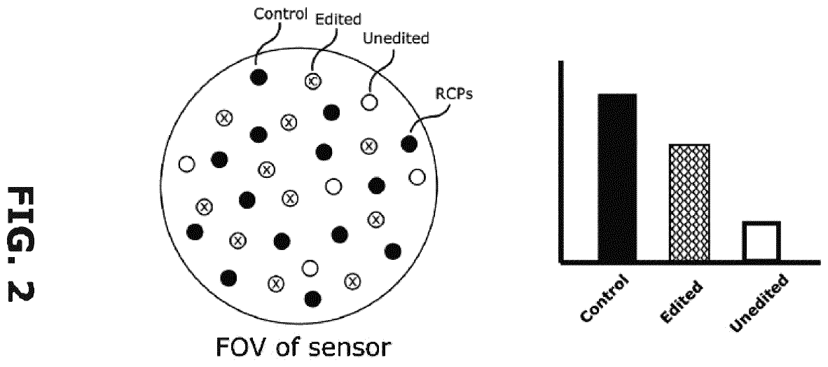

In the figures, the following reference numbers have been used:
1. Control sequence
2. Unedited sequence site
3. Edited sequence site
4. Padlock probe for control sequence
5. Padlock probe for unedited sequence
6. Padlock probe for edited sequence
7. Gap-fill probe for control sequence
8. Gap-fill probe for unedited sequence
9. Gap-fill probe for edited sequence
10. Selector probe for control sequence
11. Selector probe for unedited sequence
12. Selector probe for edited sequence
13. Gap-fill probe hybridization site
14. Target strand with single nucleotide polymorphisms site, N
15. Ligation site
16. Padlock probe with DNA base at the 3′ end for single nucleotide polymorphism site detection
17. Alternative invader probe for single nucleotide polymorphism site detection
18. Invader flap
19. Displaced invader flap
20. Traditional invader probe for single nucleotide polymorphism site detection
21. Splint probe with RNA base at the 3′ end for single nucleotide polymorphism site detection
22. Splint ligation site
23. Nick for direct ligation, or gap for filling followed by ligation
24. Lotus probe
25. Polynucleotide target sequence
26. Trilock probe
27. Trilock linker
28. Sequencing probes with probe-specific barcode compatible with NGS chemistry
29. Polynucleotide sequence with single nucleotide polymorphism

### DETAILED DESCRIPTION OF THE INVENTION

In a particular embodiment the method of the invention allows for quantifying the efficiency of targeted gene editing methods and the identification of those gene edits, and in particular to those that use circular oligonucleotide substrates generated by probe or target circularization, and subsequent rolling circle amplification (RCA).

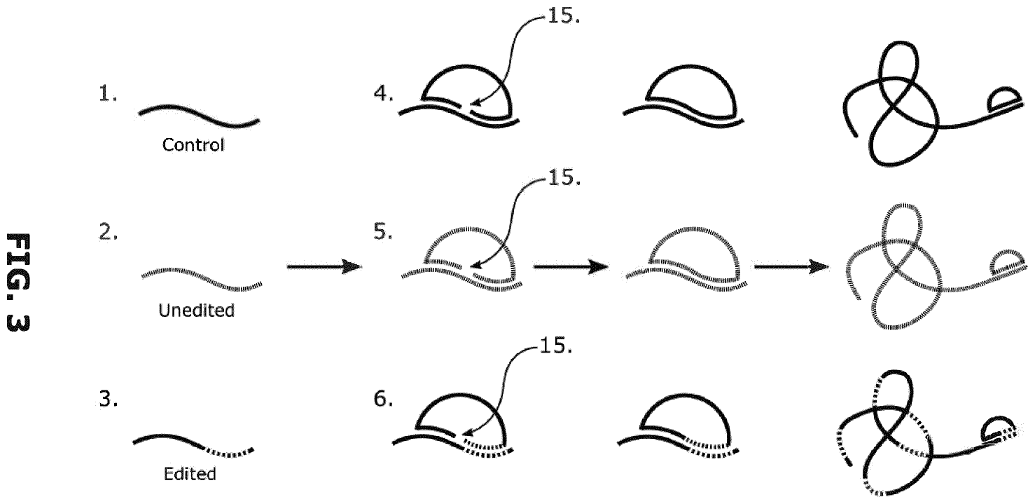

With reference to the schematics shown in FIGS. 2, 3, 4, 5, 6, 7, 8 and 9 the methods here include generating and quantifying populations of differentially labeled amplified single molecules from: a control gene sequence (1), unedited target site sequence (2) and edited target site sequence (3). By quantifying and calculating the ratio of labeled amplicons from individual populations, the efficiency of a gene editing technique can be measured with high precision. In particular, amplicons are generated through rolling circle amplification reactions of circular oligonucleotide substrates generated by highly specific multiplex target sequence recognition reactions.

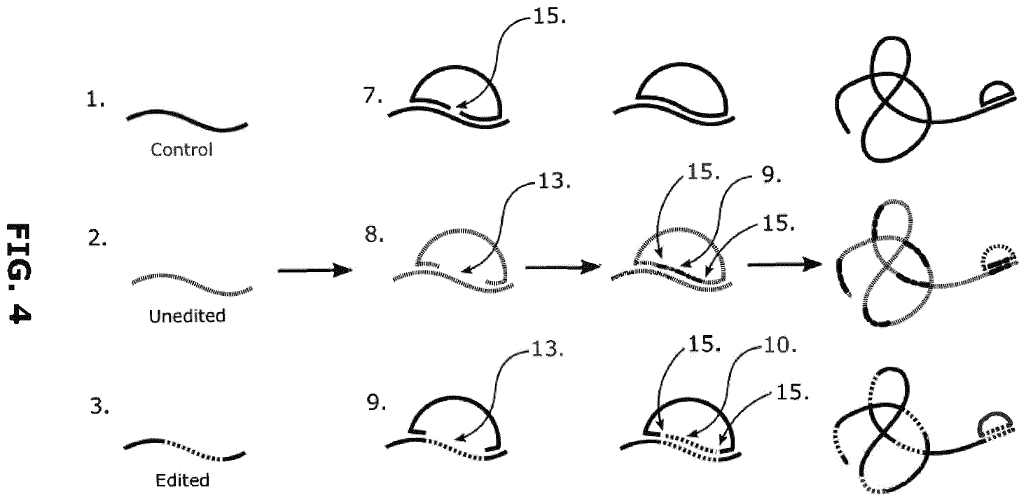

In a particular method depicted by FIG. 3, circular oligonucleotides are generated by the specific hybridization and ligation of so-called padlock probes on their respective targets. Wherein, said probes are designed to contain differential sequences that allow to differentiate between the control, edited and the unedited gene sequences. After subsequent RCA reaction, multiple complementary copies of the original probe are generated. Resulting single molecule amplicons are differentially labeled by hybridizing labeled oligonucleotide tags corresponding to those originally encoded within each probe. This generates populations of differentially labeled amplicons coming from the control, edited and unedited gene sequences site, respectively.

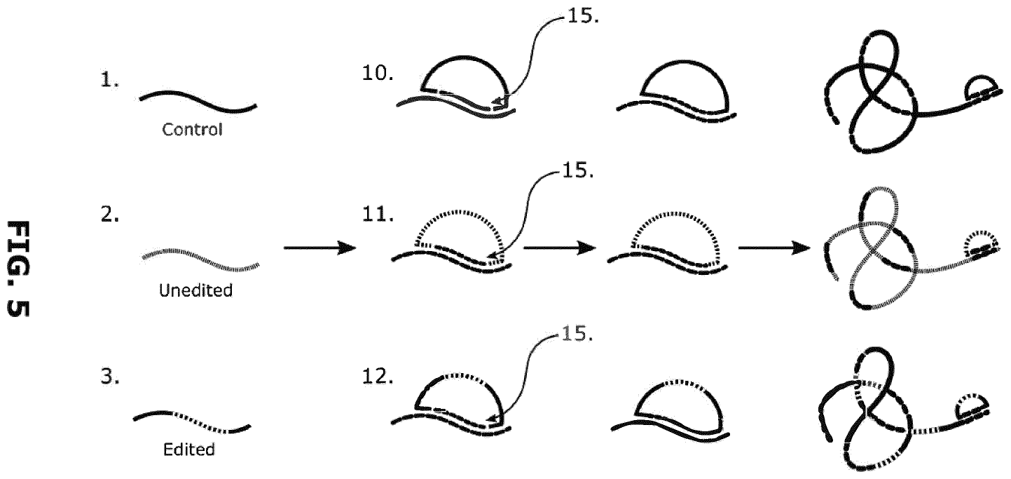

In another particular method according to the invention as depicted in FIG. 4, circular substrates for RCA are generated by the specific hybridization and ligation of so-called oligonucleotide Gap-fill probes on their respective targets. Wherein, said probe arms (i) are designed to flank the gene editing site after hybridization, thus leaving a gap on the editing site. Said gap is filled by hybridization and ligation of oligonucleotides complementary to the sequence contained within the gap thus forming circular oligonucleotide molecules. After subsequent RCA reaction, multiple complementary copies of the filled probe are generated. Resulting single molecule amplicons are differentially labeled by hybridizing labeled oligonucleotides complementary to those that originally fill the gap and to the joint arms of the Gap-fill probe. Where, gap filled labeled events will correspond to the unedited gene sequence and non-filled events will correspond to edited sequence.

In another embodiment of the method according to the invention as depicted in FIG. 4, circular substrates for RCA are generated by the specific hybridization, ligation and polymerization of so-called Gap-fill probes on their respective targets. Wherein, said probes arms (i) are designed to flank the gene editing site after hybridization, thus leaving a gap on the editing site. Said gap is filled by polymerization and ligation by the concerted action of a polymerase and a ligase, thus forming circular oligonucleotide molecules. After subsequent RCA reaction, multiple complementary copies of the filled probe are generated. Resulting single molecule amplicons are differentially labeled by hybridizing labeled oligonucleotides complementary to the expected gap sequence and to the joint arms of the Gap-fill probe. Where, gap filled labeled events will correspond to the unedited gene sequence and non-filled events will correspond to edited sequence.

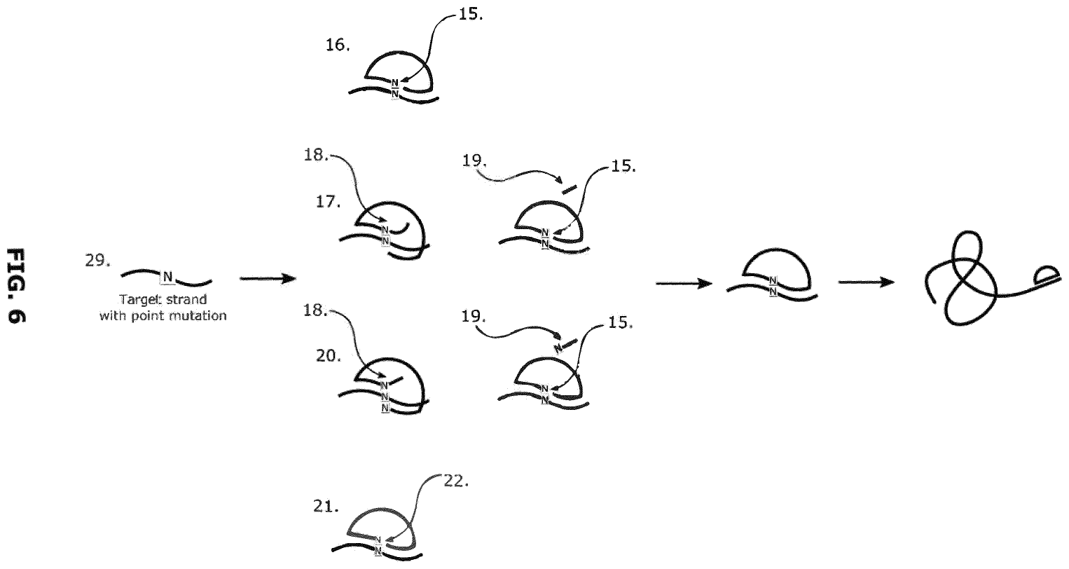

In another embodiment of the method of the invention according to the invention as depicted in FIG. 5, circular substrates are generated by circularizing the gene editing site region, where genomic DNA from edited cells is fragmented by, for instance, restriction endonucleases and subsequently denatured. A so-called selector probe, complementary to the joint ends of the fragmented gene region is used to circularize the gene edited region. After exonucleolytic digestion of non-circular targets, RCA is performed thus generating multiple copies of the gene edited region. Resulting single molecule amplicons are differentially labeled by hybridizing labeled oligonucleotides complementary to the control, unedited gene sequence and edited sequence and thus quantified to determine the ratio of editing efficiency.

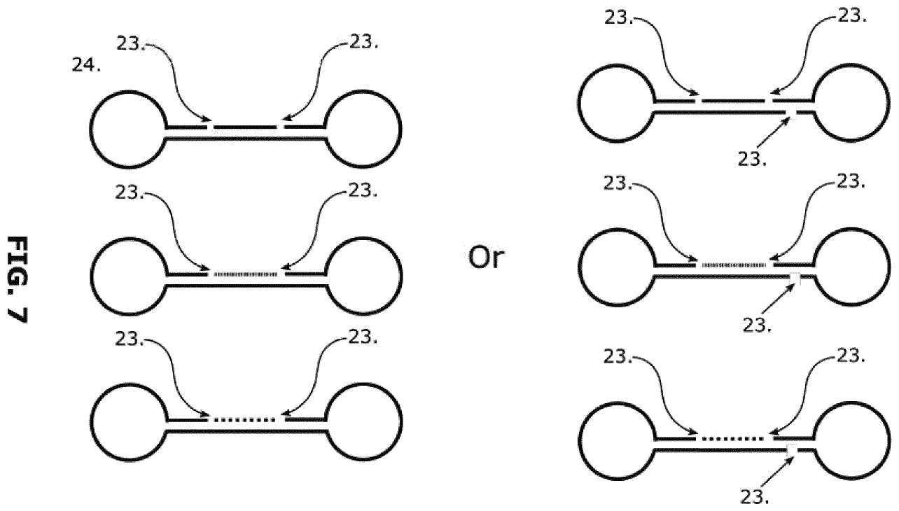

In another embodiment of the method of the invention according to the invention as depicted in FIG. 6, a polynucleotide target sequence carrying a single nucleotide polymorphism can be targeted by padlock probes carrying the specific base on the 3′ end (16), alternative invader probe (17), traditional invader probes (20) or a splint probe (21). This design offers high specificity as only in the event of perfectly matching sequences these probes can be ligated enabling to distinguish very close polynucleotide sequences.

In another embodiment of the method according to the invention as depicted in FIG. 7, a polynucleotide target sequence is fragmented into specific fragments that can subsequently hybridize to their respective lotus probe which in turn can after sealing the nick with a ligase or using a gap-fill mechanism be circularized and serve as a template for the RCA reaction.

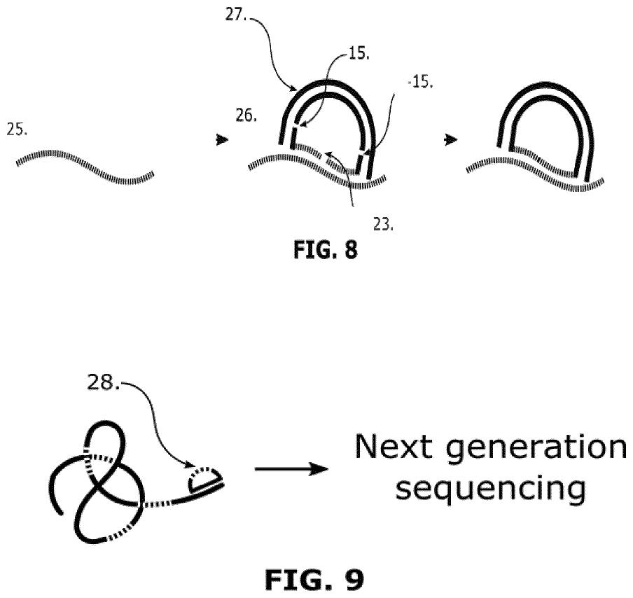

In another embodiment of the method of the invention as depicted in FIG. 8, trilock probes are used to generate a circular substrate by specifically binding to a polynucleotide sequence. The two probe arms complementary to the polynucleotide target can either be perfectly matching to create a nick site that can be sealed with a ligase, or create a gap that can be filled by a polymerase before being sealed by a ligase to subsequently serve as a template for the RCA reaction.

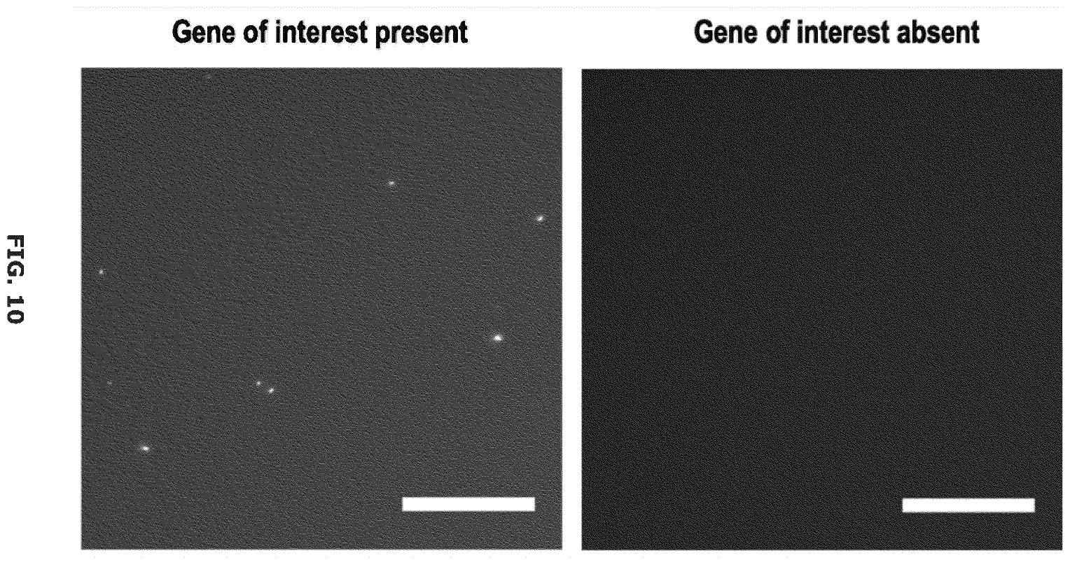

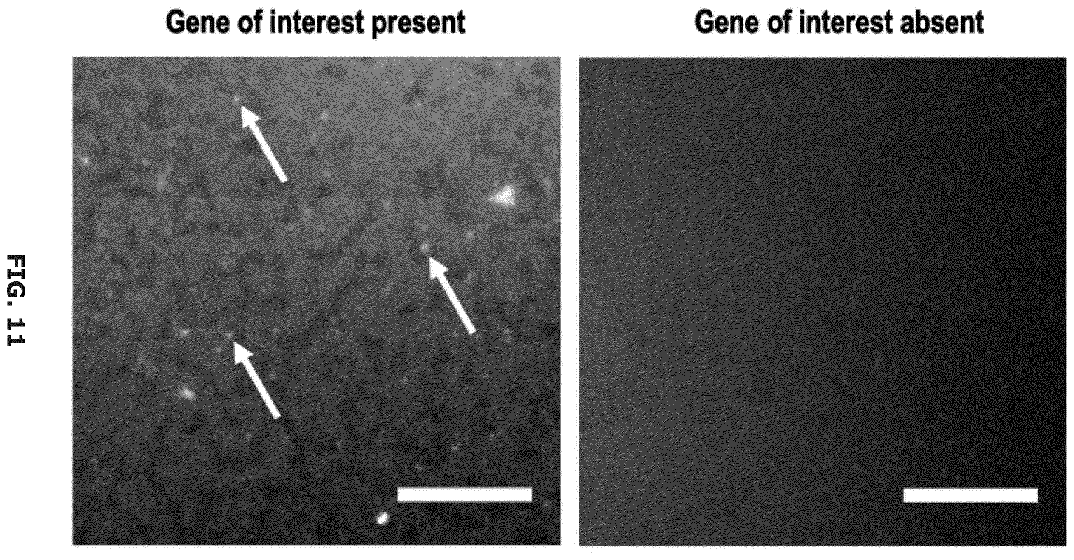

In another embodiment according to the invention as depicted in FIG. 9, the probes presented in FIGS. 2, 3, 4, 5, 6, 7 and 8 are designed in a way to contain a barcode compatible with NGS chemistry. This allows to use the generated RCPs to be not only identified and quantified by direct measurement but also by DNA sequencing.

In a particular embodiment the method may have the following steps:
DNA extraction from sample subjected gene editing experiment
DNA denaturation
Probe hybridization and ligation (this is a mix that includes probes for control, edited and non-edited)
RCA
RCP labeling
RCP immobilization in membrane
Fluorescence microscopy
Image processing and quantification of resulting spots
SEQUENCES

Padlock Probe RPP 30
SEQ ID NO: 1
PO4-TTGTTGAGTGTTGGCGTGTATGCAGCTCCTCAGTAATAGTGTCTT
ACATTTAGCATACATCGTCGCGTGCATAACCAGGCCA
Detection Oligonucleotide
SEQ ID NO: 2
TCCTCAGTAATAGTGTCTTACTTTT-CY3
PADLOCK PROBE GAPDH
SEQ ID NO: 3
PO4-AAGGGTGCAGCTGAGGTGTATGCAGCTCCTCAGTAATAGTGTCTT
ACATTTAGCATACATCGTCGCGAGCGTTTTCTCCCTA
PADLOCK PROBE NRXN1 UNEDITED
SEQ ID NO: 4
PO4-CGGCGGCCGCCTGCAGTGTATGCAGCTCCTCAGTAATAGTGTCTT
ACGGGCCTTATTCCGGTGCTATGCTGATTCTGACGCG
PADLOCK PROBE NRXN1 REFERENCE
SEQ ID NO: 5
PO4-AATAAGGGTCCCGAGGTGTATGCAGCTCCTCAGTAATAGTGTCTT
ACAGAGAGTAGTACTTCCGACTACACCGTGACGAAGA
CAPTURE OLIGONUCLEOTIDE
SEQ ID NO: 6
BIOTIN-TTTTTCCTCAGTAATAGTGTCTTAC
DETECTION OLIGONUCLEOTIDE CY3
SEQ ID NO: 7
ATTTAGCATACATCGTCGCG-CY3
DETECTION OLIGONUCLEOTIDE FITC
SEQ ID NO: 8
GGGCCTTATTCCGGTGCTATTTTTT-FITC
PADLOCK PROBE ACTB REFERENCE
SEQ ID NO: 9
PO4-TTGTAGAAGGTGTGGGTGTATGCAGCTCCTCAGTAATAGTGTCTT
ACGGGCCTTATTCCGGTGCTATGCCACACGCAGCTCA
PADLOCK PROBE ACTB EDITED
SEQ ID NO: 10
PO4-CCTTCACCGTTCCAGGTGTATGCAGCTCCTCAGTAATAGTGTCTT
ACATTTAGCATACATCGTCGCGAACCGACTGCTGTCA
PADLOCK PROBE NRXN1 SNP
SEQ ID NO: 11
PO4-CGGCGGCCGCCTGCAGTGTATGCAGCTCCTCAGTAATAGTGTCTT
ACGGGCCTTATTCCGGTGCTATGCTGATTCTGACGCT
PADLOCK PROBE ME28S:A2486 REFERENCE
SEQ ID NO: 12
PO4-TCAACACCGCTGGCAGTGTATGCAGCTCCTCAGTAATAGTGTCTT
ACGGGCCTTATTCCGGTGCTATCGGAGACAGAGAGCT
PADLOCK PROBE ACTB REFERENCE 2
SEQ ID NO: 13
PO4-TTGTAGAAGGTGTGGGTGTATGCAGCTCCTCAGTAATAGTGTCTT
ACAGAGAGTAGTACTTCCGACTGCCACACGCAGCTCA
PADLOCK PROBE ACTB REFERENCE MUT
SEQ ID NO: 14
PO4-TTGTAGAAGGTGTGGGTGTATGCAGCTCCTCAGTAATAGTGTCTT
ACATTTAGCATACATCGTCGCGGCCACACGCAGCTCG
DETECTION OLIGONUCLEOTIDE ATTO647
SEQ ID NO: 15
AGAGAGTAGTACTTCCGACT-Atto647
EXTERNAL PRIMER
SEQ ID NO: 16
TACTGAGGAGCTGCATAC*A*C
‘*’ in front of a base denotes a phosphorothioate
modification

### EXAMPLES

### Example 1

Generation of RCPs from Human DNA

RCPs illustrated in FIGS. 2 and 3 were generated from human DNA using a padlock probe ligation reaction. For this, 1 μg of human DNA (Sigma Aldrich) was fragmented in a total of 10 μL using 10 U of MscI (NEB) in 1× CutSmart buffer (NEB) at 37° C. for 30 min.

Subsequently, the reaction was inactivated and the DNA denatured by heating the reaction to 95° C. for 10 min before snap cooling it on ice. Next, the ligation mixture was prepared which was composed of 100 pM (of each) padlock probe (PO₄-TTGTTGAGTGTTGGCGTGTATGCAGCTCCTCAGTAATAGTGTCTTACATTTAGCATACATCGTCG CGTGCATAACCAGGCCA, SEQ ID NO: 1) (IDT), Tth ligase buffer (20 mM Tris-HCl (pH 8.3) (Thermo Fisher Scientific), 25 mM KCl (Thermo Fisher Scientific), 10 mM MgCl₂ (Thermo Fisher Scientific), 0.5 mM NAD (Thermo Fisher Scientific), 0.01% Triton® X-100 (Sigma Aldrich)), 0.2 μg/μL BSA (Thermo Fisher Scientific), 0.68 mM ATP (Thermo Fisher Scientific) and 5 U Tth DNA ligase (Blirt S. A) in a final volume of 20 μL. The mixture was incubated at 95° C. for 1 min followed by 50° C. for 40 min. The resulting circles from this reaction were mixed in phi29 DNA polymerase buffer (50 mM Tris-HCl (pH 8.3), 10 mM MgCl₂, 10 mM (NH₄)₂ SO₄) containing 0.2 μg/μL BSA, 125 μM dNTPs (Thermo Fisher Scientific) and 8 U of phi29 DNA polymerase (Monserate) in a total volume of 30 μL. The amplification mixture was incubated at 37° C. for 3 h followed by 65° C. for 2 min. RCPs were fluorescently labelled by adding 30 μL of hybridization buffer (1.4 M NaCl (KI Substrat), 0.01% TWEEN 20 (Sigma Aldrich), 20 mM Tris-HCl (pH 8) and 20 mM EDTA (KI Innovation)) containing 5 nM detection oligonucleotide (TCCTCAGTAATAGTGTCTTACTTTT-Cy3, SEQ ID NO: 2) (IDT) for 2 min at 75° C. and 15 min at 55° C.

RCP Detection on Slide

To visualize the RCPs, 10 μL of the hybridization reaction were put on a Superfrost slide (ThermoFisher) and spread on the surface using a 24×24 mm Menzel Glaser coverslip (VWR). The slide was incubated at room temperature for 5 min to allow the RCPs to attach to the slide. After incubation, five fields of view within the 24×24 mm coverslip were imaged with a Zeiss Axio Imager Z2 epifluorescence microscope with a 20× magnification objective with a field of view of 0.75×0.75 μm. Resulting RCPs were quantified using a custom-built pipeline using CellProfiler software. Exemplary images are shown in FIG. 10.

RCP Detection on Enrichment Sample Analysis Device

A sample analysis device as used for the enrichment in FIG. 3 was manufactured by Aline, Inc. (U.S.). The filter membrane was a Protran™ NC Nitrocellulose membrane with a 0.1 μm pore size (GE Healthcare Lifesciences), the absorption layer was a cellulose fiber sample pad sheet (Meck-Millipore), the spacing layer was in the form of pressure sensitive adhesive (Aline) and the liquid-impermeable layer was of polyethylene terephthalate (Aline). The sample receiving wells had a diameter of 1.5 mm. The sample analysis device was manufactured to have dimensions of a standard microscope slide 25×75 mm with ten sample receiving wells arrayed over the sample analysis device. Labelled dilutions of RCPs were applied onto the sample receiving wells embedded in the sample analysis device with a diameter of 1.5 mm. 10 μL of the labelled RCPs were applied onto the sample receiving wells. The applied liquid samples were let to form a large droplet on top of the sample receiving wells. After approximately 4 min, all the liquid sample had been wicked through the filter membrane. The sample receiving wells were imaged with a Zeiss Axio Imager Z2 epifluorescence microscope with a 20× magnification objective with a field of view of 0.75×0.75 μm. Resulting RCPs were quantified using a custom-built pipeline using CellProfiler software. Exemplary images are shown in FIG. 11.

### Example 2

Principle and Analytical Capability of the Invention

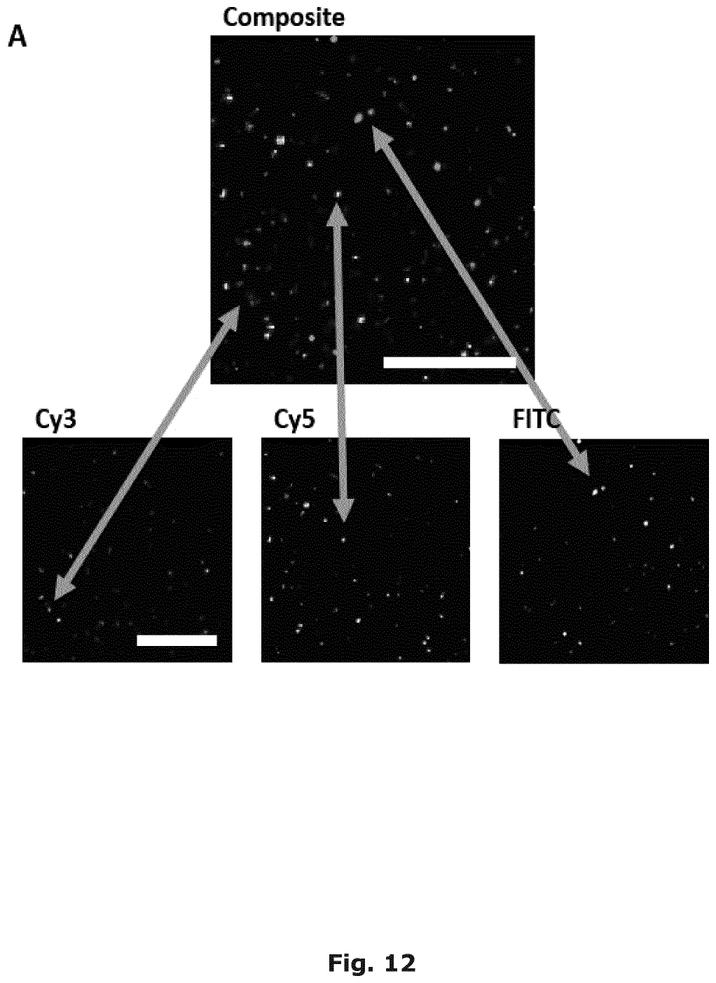

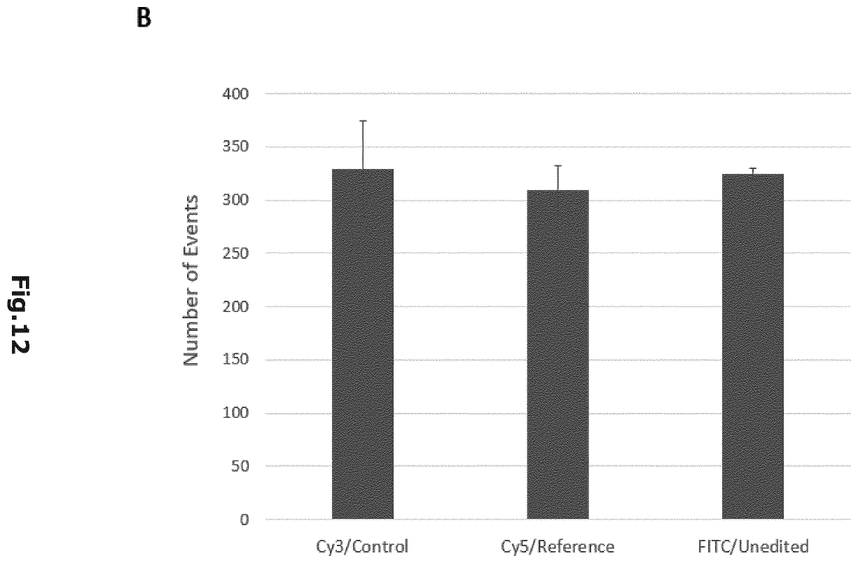

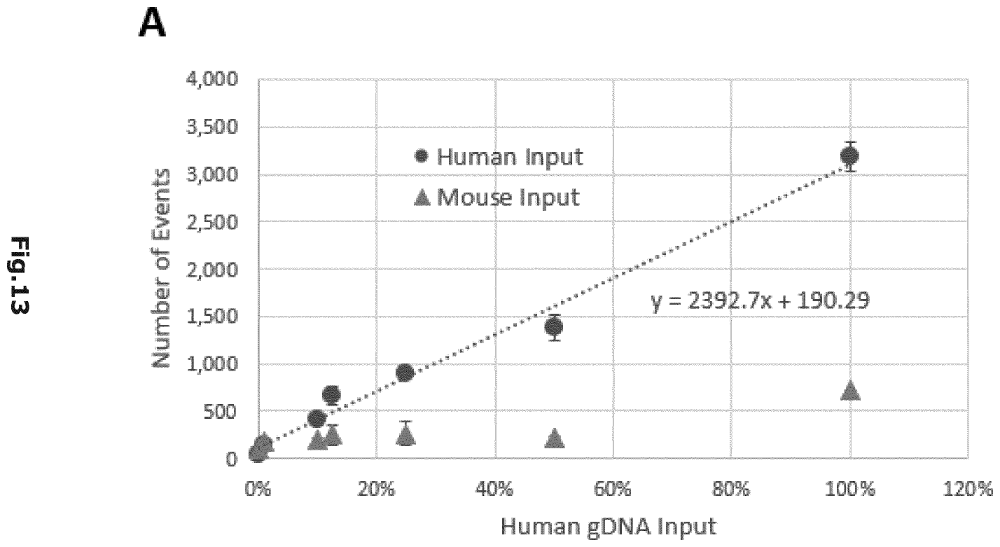

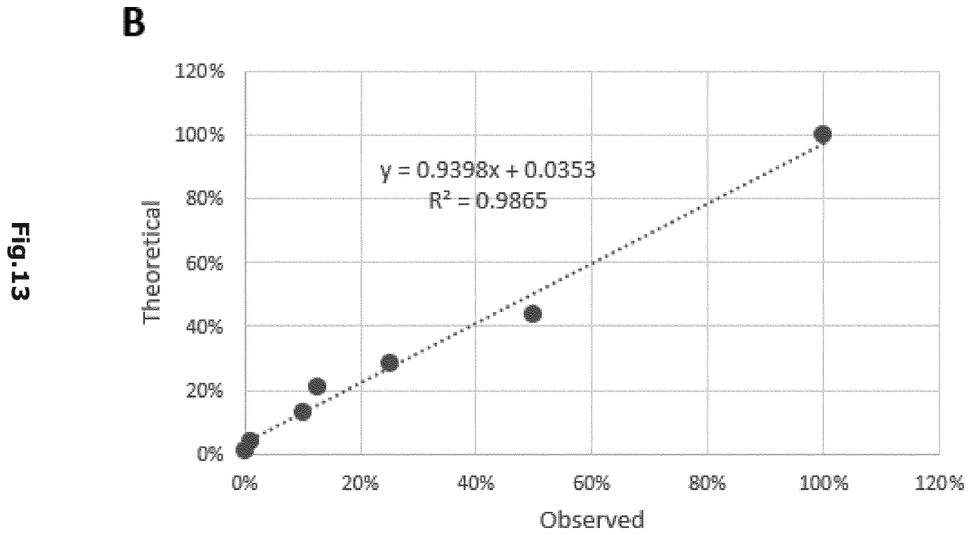

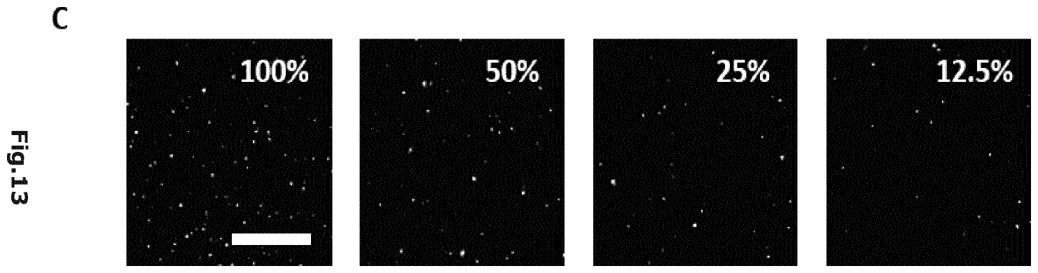

This example demonstrates the principle and the analytical capabilities using the invention to digitally determine the number of sequences present in a sample. This example is illustrated in FIG. 12.

Generation of RCPs

RCPs generated for FIG. 12 were prepared from human and mouse DNA with a constant amount (1 μg) of mouse DNA across conditions and decreasing amount of human DNA (1 μg, 500 ng, 100 ng and 1 ng respectively). For this, human (Sigma Aldrich) and mouse (Sigma Aldrich) DNA was fragmented in a total of 20 μL using 15 U of AluI (NEB) in 1× Tth buffer (Blirt) at 37° C. for 5 min. Next, the ligation mixture was prepared which was composed of 1 nM (f each) padlock probes (SEQ ID NO: 3; SEQ ID NO: 4; SEQ ID NO: 5) (IDT), Tth ligase buffer (Blirt) and 7.5 U Tth DNA ligase (Blirt) in a final volume of 30 μL. The mixture was incubated at 98° C. for 10 min followed by 55° C. for 45 min. The resulting circles from this reaction were mixed in Tth buffer (Blirt) with 24 U of Exonuclease I (NEB), 0.8 mM dNTPs, 5 nM external primer (SEQ ID NO: 16) (IDT) 6 μg BSA (Thermo Fisher Scientific) and 12 U of phi29 DNA polymerase (Blirt) in a total volume of 40 μL. The amplification mixture was incubated at 37° C. for 3 h followed by 65° C. for 2 min.

Labelling of RCPs

RCPs were fluorescently labelled by adding 10 μL of hybridization buffer (0.7 M NaCl (KI Substrat), 0.005% TWEEN 20 (Sigma Aldrich), 10 mM Tris-HCl (pH 8) and 10 mM EDTA (KI Substrat)) containing 5 nM (of each) detection oligonucleotides (SEQ ID NO: 6; SEQ ID NO: 7; SEQ ID NO: 8; SEQ ID NO: 15) (IDT) for 2 min at 75° C. and 20 min at 55° C.

Capturing RCPs on Beads

The resulting labelled RCPs were captured on Dynabeads™ MyOne™ Streptavidin T1 (Thermo Fisher Scientific). Dynabeads™ MyOne™ Streptavidin T1 beads are superparamagnetic beads having a diameter of 1 μm. For this, the beads were prepared according to the manufacturer's instructions and subsequently added to the RCP solution at a concentration of 0.125 μg/μL. The capture reaction was incubated at 37° C. for 20 min and the bead subsequently washed once in washing buffer and resuspended in the same.

Imaging of RCPs

To visualize the resulting bead-bound RCPs, 10 μL of the capture reaction were put on Superfrost glass slide (Thermo Fisher Scientific). A 2 mm circular magnet (Supermagnete) was attached to a cell counter slide (Bio-Rad) to allow the local concentration of the bead-bound RCPs on a small surface area. The slide was incubated at room temperature for 5 min to allow the beads to be enriched. After incubation, the slide was imaged with an Olympus IX73 inverted fluorescence microscope with a 20× magnification objective and a field of view of 0.65×0.65 μm².

RCP Quantification

The resulting images were analyzed using a custom-made pipeline in the CellProfiler software (version 4.1.3; https://cellprofiler.org by the Broad Institute and initially published by Lamprecht et al. CellProfiler: free, versatile software for automated biological image analysis, Biotechniques (2007); 42(1):71-75). The pipeline consisted of image enhancement and object identification with manual thresholding.

In conclusion, the results confirm the capability of the method using padlock probes and RCA to correctly quantify the presence and quantity of genes of interest. In this example, all three genes have one allele in the genome and should therefore show a similar number of RCPs/events. This is exactly what the graph in FIG. 12 shows.

### Example 3

Analytical Capability of the Invention

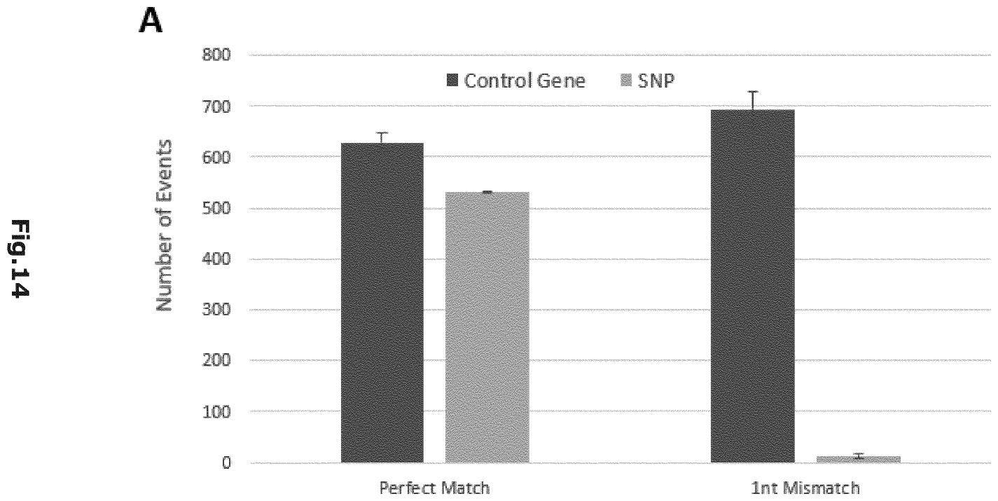

This example demonstrates the analytical capabilities using the invention to determine the percentage of a sequence interest present in a complex sample. This example is illustrated in FIG. 13.

Generation of RCPs

RCPs generated for FIG. 13 were prepared from human and mouse DNA with a constant amount (1 μg) of mouse DNA across conditions and decreasing amount of human DNA (1 μg, 500 ng, 100 ng and 1 ng respectively). For this, human (Sigma Aldrich) and mouse (Sigma Aldrich) DNA was fragmented in a total of 20 μL using 15 U of AluI (NEB) in 1× Tth buffer (Blirt) at 37° C. for 5 min. Next, the ligation mixture was prepared which was composed of 1 nM (of each) padlock probes (SEQ ID NO: 9; SEQ ID NO: 10) (IDT), Tth ligase buffer (Blirt) and 7.5 U Tth DNA ligase (Blirt) in a final volume of 30 μL. The mixture was incubated at 98° C. for 10 min followed by 55° C. for 45 min. The resulting circles from this reaction were mixed in Tth buffer (Blirt) with 24 U of Exonuclease I (NEB), 0.8 mM dNTPs, 5 nM external primer (SEQ ID NO: 16) (IDT) 6 μg BSA (Thermo Fisher Scientific) and 12 U of phi29 DNA polymerase (Blirt) in a total volume of 40 μL. The amplification mixture was incubated at 37° C. for 3 h followed by 65° C. for 2 min.

Labelling of RCPs

RCPs were fluorescently labelled by adding 10 μL of hybridization buffer (0.7 M NaCl (KI Substrat), 0.005% TWEEN 20 (Sigma Aldrich), 10 mM Tris-HCl (pH 8) and 10 mM EDTA (KI Substrat)) containing 5 nM (of each) detection oligonucleotides (SEQ ID NO: 6; SEQ ID NO: 7; SEQ ID NO: 8) (IDT) for 2 min at 75° C. and 20 min at 55° C.

Capturing, imaging and quantification was performed as described in Example 2.

In conclusion, there is a strong correlation and agreement in the number of observed RCPs as a ratio between the human reference and the different dilutions and the theoretically expected ration (in percent). The result is a simplified way to digitally quantify the presence or absence of DNA sequences when compared to competitive methods, such as digital PCR.

### Example 4

Analytical Capability of the Invention in Terms of Specificity

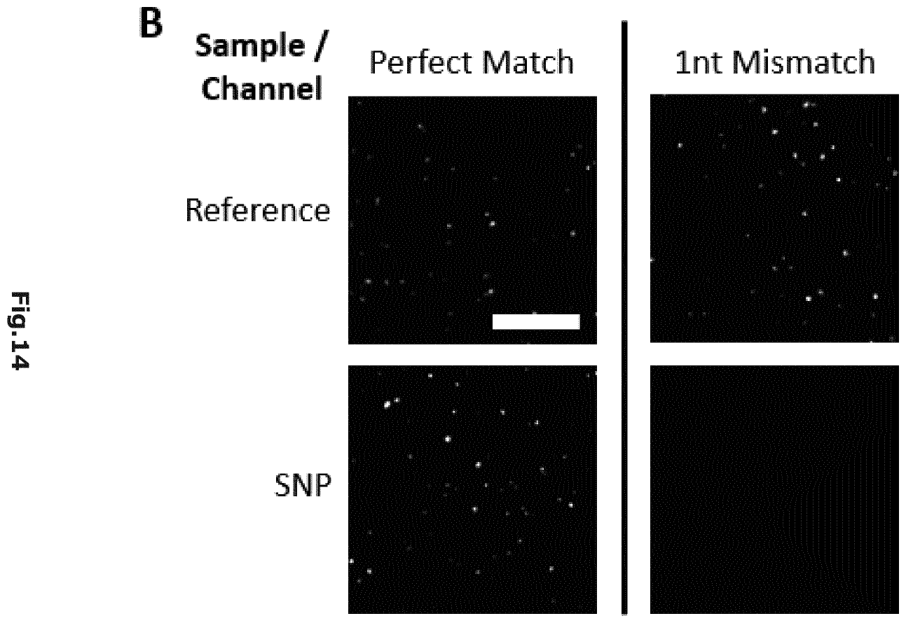

This example demonstrates the analytical capabilities in terms of specificity using the invention to distinguish between a single nucleotide difference. This example is illustrated in FIG. 14.

Generation of RCPs

RCPs generated for FIG. 14 were prepared from 1 μg of human DNA (Sigma Aldrich). For this, human DNA was fragmented in a total of 20 μL using 15 U of AluI (NEB) in 1× Tth buffer (Blirt) at 37° C. for 5 min. Next, the ligation mixture was prepared which was composed of 1 nM padlock probe (SEQ ID NO: 3) as well as either 1 nM padlock probe (SEQ ID NO: 4) as a perfect match or 1 nM padlock probe (SEQ ID NO: 11) (IDT) as a 1 nucleotide mismatch at the 3′ end, Tth ligase buffer (Blirt) and 5 U Tth DNA ligase (Blirt) in a final volume of 20 μL. The mixture was incubated at 55° C. for 20 min. The resulting circles from this reaction were mixed in phi29 DNA polymerase buffer (Blirt) containing 6 μg BSA, 0.8 mM dNTPs (Thermo Fisher Scientific), 5 nM external primer (SEQ ID NO: 16) (IDT) and 12 U of phi29 DNA polymerase (Blirt) in a total volume of 30 μL. The amplification mixture was incubated at 37° C. for 2 h followed by 65° C. for 2 min.

Labelling of RCPs

RCPs were fluorescently labelled by adding 30 μL of hybridization buffer (0.7 M NaCl (KI Substrat), 0.005% TWEEN 20 (Sigma Aldrich), 10 mM Tris-HCl (pH 8) and 10 mM EDTA (KI Substrat)) containing 5 nM detection oligonucleotide (SEQ ID NO: 6; SEQ ID NO: 7; SEQ ID NO: 8) (IDT) for 2 min at 75° C. and 20 min at 55° C.

Capturing, imaging and quantification was performed as described in Example 2.

In conclusion, the results confirm the high specificity of a padlock probe-based approach by being able to distinguish between a single nucleotide difference in a target sequence. In the event of a single nucleotide difference, the padlock probe cannot get ligated and become a circular template for the subsequent RCA reaction. This makes the method particularly useful for gene editing, where targeted evolution or genetic diseases are being studied. The high specificity makes this method particular superior over PCR-based approaches where the differentiation between a single nucleotide often requires primer optimization.

### Example 5

Quantification of Different DNA Sources

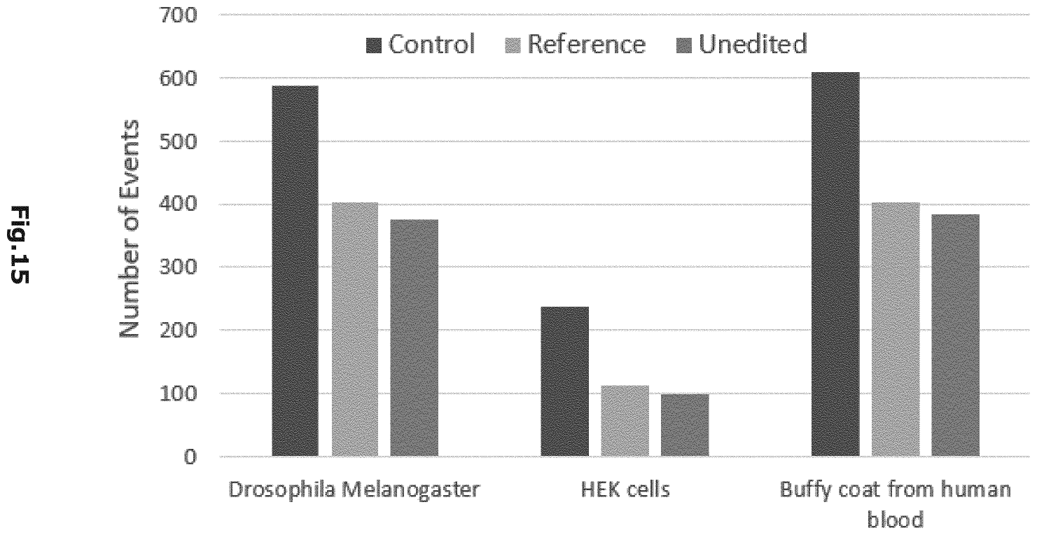

This example demonstrated the capabilities to quantify DNA independent of the organism or gene of interest. Here, DNA from Drosophila melanogaster (fruit fly), HEK cells and from human buffy coats was used as examples. This example is illustrated in FIG. 15.

Generation of RCPs

RCPs generated for FIG. 15 were prepared from different sources, namely Drosophila melanogaster, HEK cells and human buffy coat DNA (Sigma Aldrich). Each reaction was performed in a separate experiment to demonstrate the capability of the presented technology to generate and quantify RCA products from different sources and organisms. For this, the DNA was fragmented in a total of 20 μL using 15 U of AluI (NEB) in 1× Tth buffer (Blirt) at 37° C. for 15 min. Next, the ligation mixture was prepared. It was composed of 1 nM (of each) padlock probes (SEQ ID NO: 4; SEQ ID NO: 5, SEQ ID NO: 1) (IDT) for the HEK cell and buffy coat DNA, and of 1 nM padlock probes (SEQ ID NO: 12; SEQ ID NO: 13; SEQ ID NO: 14) for DNA from Drosophila, respectively, in Tth ligase buffer (Blirt) and 7.5 U Tth DNA ligase (Blirt) in a final volume of 30 μL. The mixture was incubated at 98° C. for 10 min followed by 55° C. for 45 min. The resulting circles from this reaction were mixed in Tth buffer (Blirt) with 24U of Exonuclease I (NEB), 0.8 mM dNTPs, nM external primer (SEQ ID NO: 16) (IDT), 6 μg BSA (Thermo Fisher Scientific) and 12 U of phi29 DNA polymerase (Blirt) in a total volume of 40 μL. The amplification mixture was incubated at 37° C. for 3 h followed by 65° C. for 2 min.

Labelling of RCPs

RCPs were fluorescently labelled by adding 10 μL of hybridization buffer (0.7 M NaCl (KI Substrat), 0.005% TWEEN 20 (Sigma Aldrich), 10 mM Tris-HCl (pH 8) and 10 mM EDTA (KI Substrat)) containing 5 nM (of each) detection oligonucleotides (SEQ ID NO: 6; SEQ ID NO: 7; SEQ ID NO: 8; SEQ ID NO: 15) (IDT) for 2 min at 75° C. and 20 min at 55° C.

Capturing, imaging and quantification was performed as described in Example 2.

In conclusion, the results confirm the capabilities of the method to readily detect genes of interest from different DNA sources. Furthermore, the example confirms the padlock probe set up, where a reference shows the same level of events as the unedited sequence. In case of an editing event, the ratio between reference to unedited or control to unedited allows to draw conclusions on the editing level. This makes the method easy to use for different research questions and uses within the gene editing field.

1. A method for determining the efficiency of a genetic-editing procedure, the method comprising the steps of:
(i) providing a sample from a genetic-editing procedure, the sample comprising one or more correctly-edited polynucleotide sequence and/or one or more unedited polynucleotide sequence;
(ii) performing Rolling Circle Amplification, to generate RCA-Products from the one or more polynucleotide sequences in the sample; and
(iii) determining the efficiency of the genetic-editing procedure based on the presence of the RCA-Products generated in step (ii).

2. The method of claim 1, wherein prior to performing Rolling Circle Amplification step (ii), the method comprises the step of generating circular single-stranded polynucleotide substrates from the polynucleotide sequences in the sample, wherein the circular single-stranded polynucleotide substrates are specific for the correctly-edited polynucleotide sequence, and for the unedited polynucleotide sequence.

3. The method of claim 1, wherein the RCA-Products are labelled with a detectable moiety, and are preferably differentially-labelled such that RCA-Products from the correctly-edited polynucleotide sequence and/or the unedited polynucleotide sequence can be distinguished.

4. The method of claim 1, wherein step (iii) comprises quantifying the RCA-Products generated in step (ii), and determining the amounts of the correctly-edited polynucleotide sequence and/or unedited polynucleotide sequence in the sample.

5. The method of claim 1, wherein step the method further comprises the step of quantifying the total number of polynucleotide sequences in the sample.

6. The method of claim 1, wherein the circular single-stranded polynucleotide substrates are generated using a first oligonucleotide probe which specifically targets the correctly-edited polynucleotide sequence, and a second oligonucleotide probe which specifically targets the unedited polynucleotide sequence.

7. The method of claim 6, wherein the first and second oligonucleotide probes are selected from the group comprising: a padlock probe; a molecular inversion probe; a gap-fill probe; a split-like probe; a Lotus probe; or a combination thereof.

8. The method of claim 6, wherein the first and second oligonucleotide probes circularise on recognition of their specific target polynucleotide sequence.

9. The method of claim 8, wherein circularisation of the first and/or second oligonucleotide probes is mediated and/or improved by one or more Joining probe; optionally wherein the one or more Joining probe is a Selector probe.

10. The method of claim 6, wherein the circular single-stranded polynucleotide substrates are formed by ligation of the first oligonucleotide probe and its specific target polynucleotide sequence, and by ligation of the second oligonucleotide probe and its specific target polynucleotide sequence; optionally wherein ligation is performed by a ligase with specific intramolecular ligation activity.

11. The method of claim 6, wherein Rolling Circle Amplification of the circular single-stranded polynucleotide substrates is initiated by the target sequence or by an amplification primer that is complementary to the circular singe-stranded polynucleotide substrate.

12. The method of claim 6, wherein the detectable moiety is selected from the group comprising: a fluorophore; a chromophore; or a combination thereof.

13. The method of claim 1, wherein step (iii) comprises immobilizing the RCA-Products on a surface.

14. The method according to claim 13, wherein the RCA-Products are immobilized on the surface by electrostatic interactions, covalent interactions, or steric interactions with said surface.

15. The method according to claim 13, wherein the surface is a glass surface or a porous membrane.

16. The method according to claim 15, wherein the porous membrane is a filter membrane, for example a porous hydrophilic membrane.

17. The method according to claim 15 wherein the RCA-Products are immobilized by filtering through the porous membrane.

18. The method according to claim 15, wherein the glass surface is modified to interact with an RCA-Product.

19. The method according to claim 1, wherein the RCA-products are flowed through a microfluidic channel so as to concentrate the sample into a single view of a microscope.

20. The method of claim 1, wherein step (iii) comprises detecting the RCA-Products by microscopy; optionally wherein microscopy is selected from the group comprising: bright-field microscopy; fluorescence microscopy; or a combination thereof.

21. The method of claim 1, wherein step (iii) comprises detecting the RCA-Products by DNA sequencing.

22. The method according to claim 1, wherein the efficiency of the genetic-editing procedure is determined based on the relative amounts of the correctly-edited polynucleotide sequence and the unedited polynucleotide sequence in the sample.

23. The method according to claim 13, wherein the method comprises the step of attaching the RCA-products to magnetic beads to provide bead-bound RCA-products.

24. The method according to claim 23, wherein the RCA-products are immobilized by providing a magnetic source so as to attract the bead-bound RCA-products to a position on the surface.

25. A method for identifying the product of a genetic-editing procedure, the method comprising the steps of:
(i) providing a sample from a genetic-editing procedure, the sample comprising one or more correctly-edited polynucleotide sequence and/or one or more unedited polynucleotide sequence;
(ii) performing Rolling Circle Amplification, to generate RCA-Products from the one or more polynucleotide sequences in the sample; and
(iii) determining the products of the genetic-editing procedure based on the presence of the RCA-Products generated in step (ii).

26. (canceled)

27. A kit of parts comprising:
a first container comprising genetic editing reagents;
a second container comprising RCA reagents; and
instructions for use of the kit in the method according to claim 1.

28. (canceled)
